(19) United States
(12) Patent Application Publication
(10) Pub. No.: US 2006/0100673 A1
(43) Pub. Date: May 11, 2006

(54) Programmable device for an implant comprising a base station and a detachable hand set

(76) Inventors: Michael Koinzer, Bergholz-Rehbruecke (DE); Klaus Brandau, Eichwalde (DE); Bernhard Gromotka, Berlin (DE)

Correspondence Address: HAHN LOESER & PARKS, LLP, One GOJO Plaza, Suite 300, AKRON, OH, 44311-1076, US

(21) Appl. No.: 10/526,057

(22) PCT Filed: Aug. 28, 2003
(86) PCT No.: PCT/EP03/09518

§ 371 Date: Aug. 4, 2005

(30) Foreign Application Priority Data
Aug. 29, 2002 (DE) 202136132

(51) Int. Cl. A61N 1/08

(52) U.S. Cl. 607/60; 607/30

(57) ABSTRACT

The invention concerns an external programming device for an implant comprising a transmitting and receiving unit for transmitting and receiving data to and from the implant, and a display with a display control unit which are connected to the transmitting and receiving unit. The device is made in a modular fashion from at least one autonomous hand device and at least one base device in such a device. The hand device includes the transmitting and receiving unit the display and the display control unit, a mains-independent chargeable power supply, a power supply interface and a data interface. The base device includes a second power supply interface compatible with the power supply interface of the hand device and a second data interface compatible with the data interface of the hand device. The hand device can be selectively electrically and mechanically coupled to the base device or separated from the base device and used autonomously.

### BACKGROUND OF THE INVENTION

The invention concerns an external programming device for an implant such as a cardiac pacemaker, defibrillator or the like, comprising a transmitting and receiving unit for receiving data on the part of the implant and transmitting data to the implant and a display with a display control unit which are adapted to display representations of transmitted and/or received data and are connected to the transmitting and receiving unit, and a power supply unit. The invention also concerns a hand device for a programming device of that kind as well as a base device.

Programming devices are basically known and serve for telemetric programming of implants such as cardiac pacemakers or defibrillators. Usually for that purpose, a programming head is applied to the skin of a patient so that the telemetric communication with the implant is made through the patient tissue.

The telemetric communication is preferably bidirectional so that data can also be read out of the implant such as, for example, such data as represent an intracardial electrocardiogram.

General demands on such a programming device are that on the one hand, it is to be easy to handle but on the other hand, for example, it is to readily also permit printout of Intracardial electrocardiograms.

### BRIEF SUMMARY OF THE INVENTION

The known programming devices do not satisfactorily meet those demands so that the object of the present invention is to provide a programming device which affords the physician as many possible options and functions in an ergonomic manner.

In accordance with the invention, that object is attained by a programming device of the kind set forth in the opening part of this specification, which is made up in a modular fashion from at least one autonomous hand device and at least one base device in such a way that the hand device includes the transmitting and receiving unit and as well as the display and the display control unit and a mains-independent chargeable power supply and a power supply interface and a data interface. In addition, the base device includes a second power supply interface compatible with the power supply interface of the hand device and a second data interface compatible with the data interface of the hand device. In that respect, the hand device and the base device are such that the hand device can be selectively electrically and mechanically coupled to the base device or separated from the base device and used autonomously, wherein the chargeable power supply of the hand device is to be charged up by way of the power supply interface by the base device when the hand device is coupled to the base device.

The essential advantage of a programming device which is of a modular structure in that way is that the hand device can be in the form of a small unit which is easy to manage and readily portable by a physician and which permits autonomous operation and in that respect affords the physician all essential functions.

The fact that the hand device can be coupled to the base device means that functions which are required less frequently such as for example, a printer function or the like, can be provided in the base device and thus do not put a burden on the hand device. Those functions are available immediately when the hand device is coupled to the base device. On that occasion, the hand device is advantageously automatically charged by the base device.

In a preferred embodiment, the hand device has a data memory which is connected on the one hand to the transmitting and receiving unit and is adapted for autonomous storage of data transmitted from the implant or to the implant, and which on the other hand is connected to the data interface of the hand device in such a way that data are to be at least unidirectionally transmitted from the data memory by way of the data interfaces to the base device when the hand device is coupled to the base device.

By virtue of that variant of a hand device with data memory which, particularly when the hand device is uncoupled from the base device, serves as an intermediate memory for such data which are to be printed out by way of the base device, it is readily possible to implement autonomous operation with the hand device and later, after the hand device has been coupled to the base device, to print out the data which are of interest, by way of the base device.

A particularly preferred form of a programming device of the last-mentioned kind is one in which the base device has a printer interface or a printer for printing out representations corresponding to the data in the data memory of the hand device. In that case, the hand device is preferably provided with a control unit which is connected and adapted to automatically detect a coupled condition of the hand device and in response to detection of the coupled condition to produce a communication between the transmitting and receiving unit of the hand device and the printer interface of the base device in such a way that data received on the part of the implant from the transmitting and receiving unit can be represented in real time by way of the printer or the printer interface.

The particular advantage of the last-mentioned variant is that the hand device can be used in its condition of being coupled to the base device, in such a way that all data received on the part of the transmitting and receiving unit of the hand device can be printed out directly by way of the printer of the base device, that is to say in real time.

Preferably, the hand device and the base device of the programming device each have a respective data transmitting and receiving unit for wireless data exchange between the hand device and the base device.

The advantage of data transmitting and receiving units at the hand device and at the base device is that the data interface between the hand device and the base device can operate wirelessly so that the hand device is easily portable even in the uncoupled condition from the base device and at the same time, for example, a real-time printout of data occurring in the hand device by way of the printer of the base device is possible.

In accordance with the invention, the above-stated object is also attained in a hand device for a programming device of the above-mentioned kind wherein the display is formed by a touch-sensitive display screen (touch screen).

Such a touch screen is particularly advantageous, in particular in conjunction with the hand device, for the reason that regions of the screen are to be programmed as touch-sensitive switching surfaces which can each be different according to the respective operating mode of the hand device. That saves on having to provide all required switches constantly beside the screen.

In addition, preferably the hand device is adapted to make a latching mechanical connection to the base device, wherein a release button is provided on the hand device for releasing the latching mechanical connection. A latching mechanical connection between the hand device and the base device permits the hand device to be securely fixed to the base device.

In addition, preferably the hand device has a shock triggering button which is connected to and co-operates with a control unit of the hand device and the transmitting and receiving unit in such a way that the discharge of a shock by the implant is to be triggered by way of the triggering button.

In that way, for example, a defibrillation shock from the implant can be remotely triggered with the hand device if that representation of the intracardial electrocardiogram on the hand device displays, for example, fibrillation of the heart of the patient.

In addition, a preferred hand device is one which has a programming head which is releasably connected to the hand device by way of a flexible electrical feed line and which is part of the transmitting and receiving unit of the hand device. By way of the programming head that permits a telemetric communication between the implant and the hand device by applying the programming head to the body of the patient.

The above-stated object is also attained in a base device for a programming device of the above-mentioned kind which has a main body and a mounting tilting member pivotably connected to the main body for mounting the hand device and adjustment of the angle of inclination of the display of the hand device when the hand device is in the coupled condition. That permits the programming device to be of an ergonomic configuration even in the condition in which the hand device is coupled to the base device.

Advantageously the mounting means has plug connections for the data interface and the power supply interface to the hand device.

In addition, the base device preferably includes a printer in its main body.

Details of the invention will be more fully described with reference to the specific description hereinafter and the drawings.

### BRIEF DESCRIPTION OF THE DRAWINGS

In the drawings:

FIG. 1 shows a front view of the programming device with the base device and the hand device removed,

FIG. 2 shows a rear view of the programming device with the hand device coupled to the base device,

FIG. 3 shows a diagrammatic block representation of the functional components of the hand device and the base device,

FIG. 4 shows a diagrammatic representation of a first display mode for the representation of intracardial cardiograms on the display of the hand device, and

FIG. 5 shows a representation of a second display mode for the representation of intracardial cardiograms on the display of the hand device.

### BRIEF DESCRIPTION OF THE INVENTION

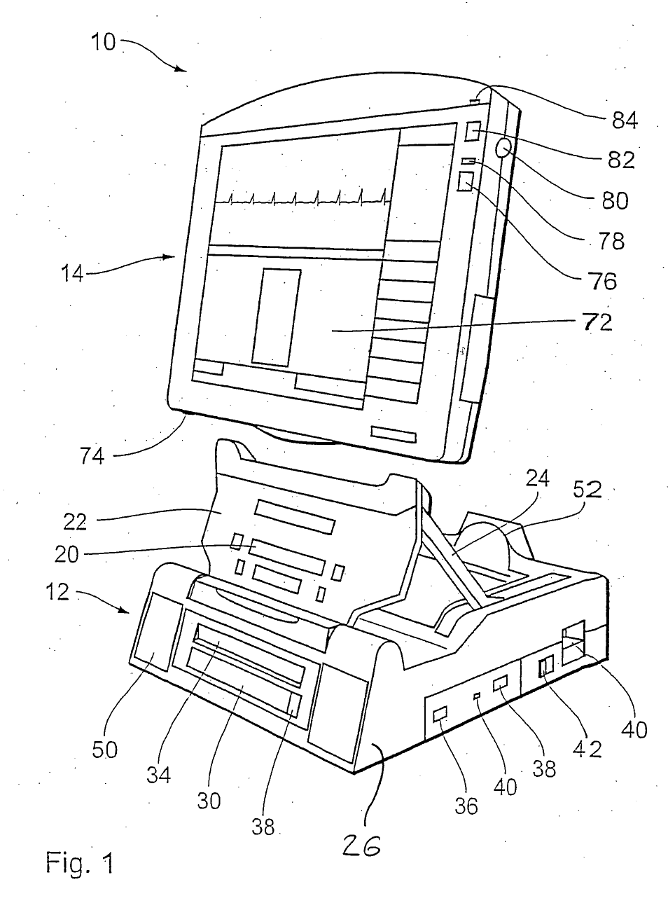

The programming device 10 shown in FIG. 1 is of a two-part modular structure and includes a base device 12 and a hand device 14.

As can be seen from FIG. 1 the base device 12 and the hand device 14 are to be separated from each other.

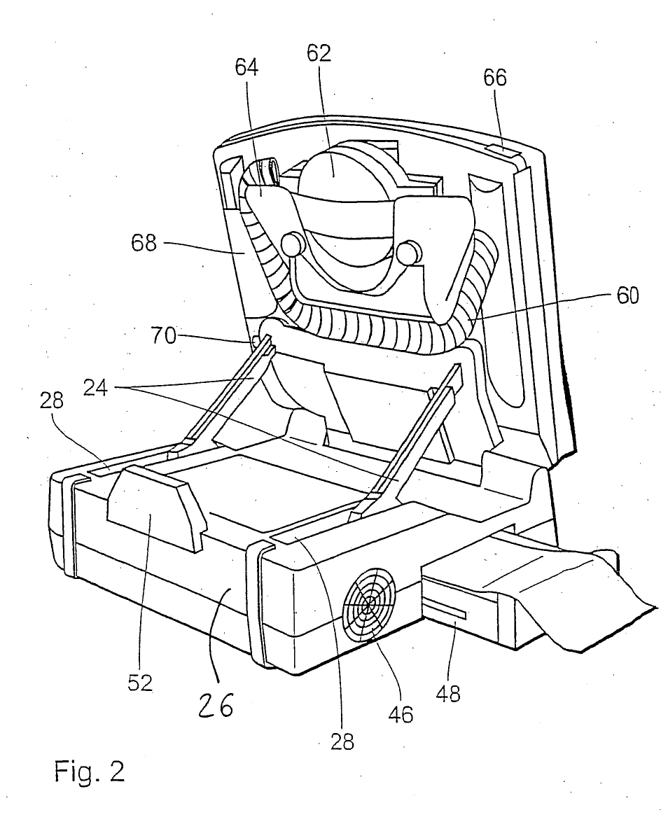

FIG. 2 is a rear view of the base device 12 and the hand device 14 in a condition in which the two devices are coupled together.

For electrically coupling the hand device 14 to the base device 12, the latter has a central plug connection 20. That central plug connection 20 is disposed on the front side of a mounting tilting member 22 which also serves for mechanically mounting the hand device 14. The mounting tilting member 22 is pivotably mounted to a front side of the base device 12 and is supported by two supports 24, at its side remote from its pivotal mounting to the base device 12. Those two supports 24 are pivotably attached to the mounting tilting member 22. At their respective other end the supports 24 are longitudinally displaceably connected to a main body 26 of the base device 12 so that adjusting of the angle between the main body 26 of the base device 12 and the mounting tilting member 22 is possible by longitudinal displacement of the supports 24 with respect to the main body 26 of the base device 12. For that purpose the main body 26 of the base device 12 has longitudinal guides 28 for the supports 24.

The following further components are disposed in the main body 26 of the base device 12: a carrying handle 30 which is lockable in the main body 26 and which issues somewhat from the main body 26 under a spring force by pressing a button 34. In addition, the main body 26 includes a CD-ROM drive 34 as well as a USB interface 36, a serial interface 38, a mains power connection 40, an on switch 42 which is to be actuated when the battery is discharged, an operating display 44, a fan with fan cover 46 and a printer with an extendable paper supply container 48. The printer is to be operated by way of a printer keyboard 50.

There is also a support leg 52 on which the coupled hand device 14 can rest when the mounting tilting member 22 is laid entirely flat.

It is also to be noted that the following signals are applied at the central plug connection: supply voltages, battery charge (the plug contacts provided for that purpose form the power supply interface), serial interface USB (Universal Serial Bus), I²C/SMBus and various control signals for mains relay, docking detection, system reset and so forth. There is also provided an expansion plug connector (not further shown here). Optional expansion modules are to be connected to a central unit (not shown) of the base device 12 by way of that expansion plug connection.

As shown in FIG. 1, the hand device 14 is to be uncoupled from the base device 12 and used autonomously. For that purpose the hand device 14 has a battery power supply disposed behind a cover 58. The hand device 14 is connected by way of an electrical feed line 60 to a programming head 62. The programming head 62 is adapted to be placed on the skin of a patient in order to make a telemetric connection which is as short as possible with an implant 100 of the patient. A suitable holder 64 is provided on the rear side of the hand device 14 for receiving the programming head 62. The arrangement also has a connecting socket for the electrical feed line 60, which in FIG. 2 is covered by the programming head 62, in order to be able to separate the programming head 62 together with the feed line 60 from the hand device 14.

Provided on the rear side of the hand device 14 is a counterpart plug connection (not shown in the Figures) which, in the coupled condition of the hand device 14 shown in FIG. 2, engages into the central plug connection 20 in the mounting member 22 of the base device 12. In the coupled condition moreover the hand device 14 is mechanically locked in per se known manner to the mounting tilting member 22. That locking action is to be released by means of a locking button 68.

The hand device 14 also has a fan disposed behind a fan cover 70.

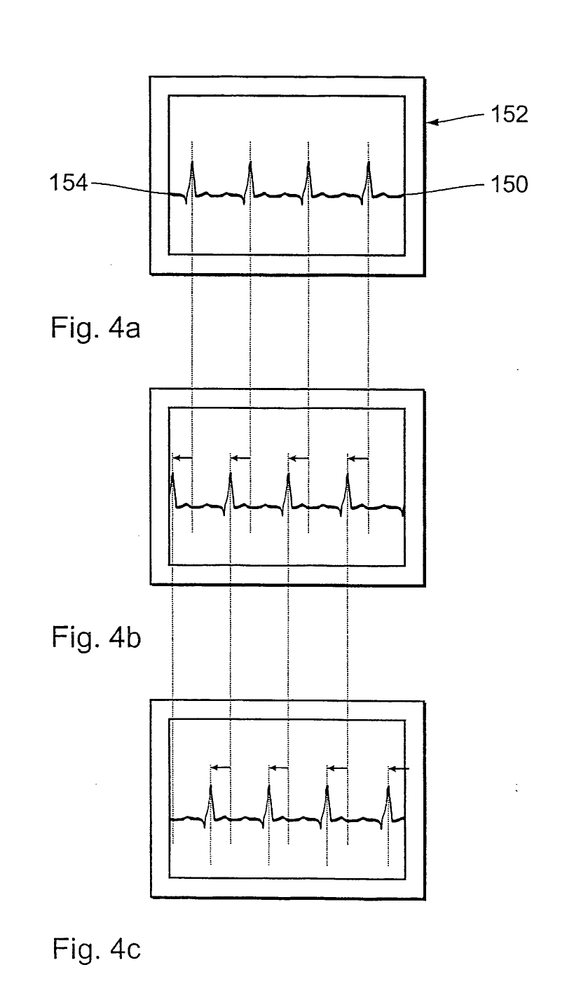

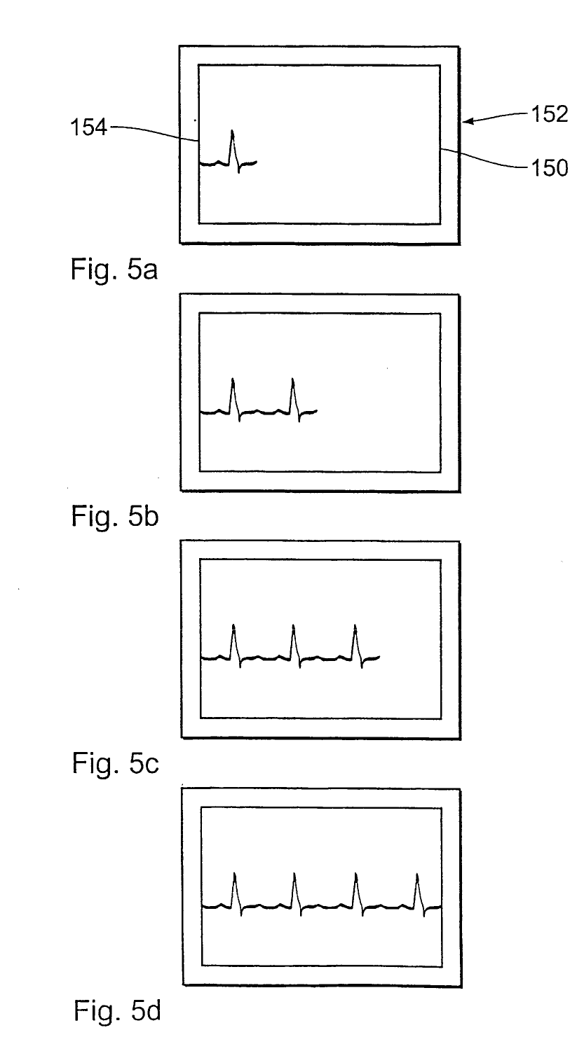

Provided on the front side of the hand device 14 as an essential component is a display 72 in the form of a touch screen. As the screen 72 is touch-sensitive, it can represent in per se known manner programmed switching surfaces which upon actuation lead to corresponding input signals from the hand device 14. Such a programmed switching surface is that for switching over between two representation modes for the representation of intracardial cardiograms which will be described in greater detail hereinafter with reference to FIGS. 4 and 5.

Further components of the hand device 14 are an emergency shock button 74 with which the implant 100 of a patient can be energized from the hand device 14 by way of the programming head 10 to deliver a defibrillation pulse.

There is also provided a charge condition button 76, upon the actuation of which the charge condition of the battery power supply of the hand device 14 is displayed. That is effected by way of a charge condition display 78.

The hand device 14 further has a socket 80 for connecting a device for recording surface electrocardiograms.

It will be appreciated that the hand device 14 also has an on switch 82 and an operating display 84.

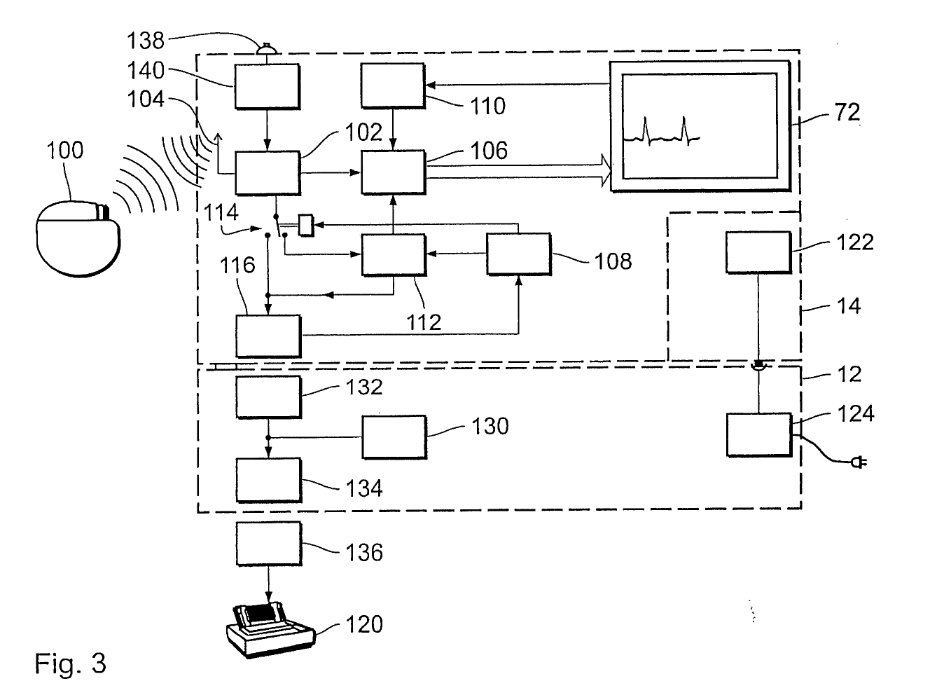

FIG. 3 shows a highly diagrammatic view of some of the internal components of the hand device 14 and the base device 12 as well as an implant 100. The hand device 14 is adapted to make a bidirectional telemetric connection between the implant 100 and the hand device 14. For that purpose the hand device 14 includes a transmitting and receiving unit 102 which, like an antenna 104, are a component part of the programming head 62. Signals on the part of the implant 100, which are recorded by the transmitting and receiving unit 102, are passed on the one hand to a display control unit 106 which actuates the display screen 72 for the display for example of electrograms intracardially recorded by the implant 100. The display control unit 106 is also connected to a central control unit 108 so that any symbol indicated by the central control unit 108, inter alia switching surfaces or text labels, can be represented on the display screen 72.

As the display screen 72 is in the form of a touch screen, it is connected to a detection unit 110 which, when the display screen 72 is touched, generates a signal corresponding to the location at which it was touched. The evaluation unit 110 is also connected to the control unit 106.

Depending on respective actuation by the central control unit 108, signals are passed from the transmitting and receiving unit 102 directly to the actuating unit 106 or to a data memory 112. In addition, the central control unit 102 can provide, by way of a switch 114, that data from the transmitting and receiving unit 102 are applied directly to a central connection plug 116 of the hand device 14. The central connecting plug 116 is connected to the central connection plug 20 of the base device 12, when the hand device 14 is coupled to the base device 12.

The central control unit 108 is also connected to the central connection plug 116 of the hand device 14 in such a way that the central control unit 108 independently detects when the hand device 14 is coupled to the base device 12. In that situation, the central control unit 108 automatically produces a data connection between the transmitting and receiving unit 102 and the central connection plug 116 in order to provide a real-time print-out of an intracardial electrocardiogram received by the transmitting and receiving unit 102, on a printer 120 of the base device 12.

For receiving the data from the hand device 14, the base device 12 has a second data interface 132 connected to a printer interface 134. There is also provided a base device data memory 130 with which data can be put into intermediate storage, between the data interface 132 and the printer interface 134. A printer 120 can be connected with its interface 136 to the base device 12, by way of the printer interface 134. In a preferred embodiment, which is illustrated in FIGS. 1 and 2 the printer is integrated into the base device 12.

In order to actuate the implant 100 under remote control by way of the hand device 14 for the delivery of a defibrillation shock, the hand device 14 has the shock triggering button 138 (emergency shock button 74 in FIG. 1) which is connected to the transmitting and receiving unit 102 of the hand device 14 by way of a suitable shock actuation unit 104.

When the hand device 14 is coupled, the battery power supply 122 of the hand device 14 is also electrically connected to a mains power supply 124 of the base device 12 in order to charge up the battery of the hand device 14.

The central control unit 108 has a program memory and is so programmed that, upon the display of intracardial electrocardiograms on the display screen 72, beside the represented electrocardiogram, there also appears a switching surface with which it is possible to switch over the display of intracardial electrocardiograms on the display screen 72. If the act of touching the switching surface for switching over the representation mode is detected by way of the evaluation unit 110 and the actuating unit 106 as well as the central control unit 108, the representation mode is switched over from a first to a second mode or vice-versa.

The two representation modes are shown in FIGS. 4 and 5. In the first representation mode shown in FIGS. 4a through 4c a respectively current signal value of the intracardial electrocardiogram is represented at a right-hand representation edge 150 of a representation window 152 on the display screen 72. All preceding signal values of the ECG are represented further to the left thereof, more specifically in such a way that the oldest represented ECG signal value is at the left-hand representation edge 154. With the representation of each new ECG signal value at the right-hand representation edge 150, all earlier ECG signal values are displaced towards the left by a display screen position. That is symbolically represented in FIGS. 4b and c. That gives the impression that the ECG displayed in the first representation mode in the display screen 72 runs continuously from right to left through the representation window 152 on the display screen 72.

In the second representation mode illustrated in FIGS. 5a through d, the electrocardiogram, starting from the left-hand representation edge 154, is continuously written along as far as the right-hand representation edge 150, insofar as each new ECG signal value is attached to the ECG signal values which have already been represented, in a condition of being displaced towards the right by a horizontal display screen position. When, as shown in FIG. 5d, the representation has reached the right-hand display screen edge 150, the display is extinguished and the procedure of progressively writing the ECG at the left-hand representation edge 154 is begun afresh.

By virtue of the fact that the display of electrocardiograms can be switched over between the two above-described representation modes, it is possible for the first time for the physician in charge of treatment to freely select the mode of representation which is respectively appropriate for observing the phenomenon in which he is interested.

When the hand device 14 is coupled to the base device 12 the physician also receives a print-out of the respective current ECG in real time. The fact that the hand device 14 can be uncoupled from the base device 12 also affords the physician the possibility of moving autonomously in space with the hand device 14 while in that case nonetheless having all essential functions and modes of representation available to him.

1. An external programming device for an implant such
comprising a transmitting and receiving unit for receiving data from the implant, and transmitting data to the implant, and
a display with a display control unit which are adapted to display representations of transmitted and/or received data and are connected to the transmitting and receiving unit, and
a power supply unit,
characterized in that
the external programming device is made up in a modular fashion from at least one autonomous hand device and at least one base device in such a way that
the hand device includes the transmitting and receiving unit and as well as the display and the display control unit and a mains-independent chargeable power supply and a power supply interface and a data interface, and
the base devices includes a second power supply interface compatible with the power supply interface of the hand device and a second data interfaced compatible with the data interface of the hand device,
in such a way that the hand device can be selectively electrically and mechanically coupled to the base device or separated from the base device and used autonomously, wherein the chargeable power supply of the hand device is to be charged up by way of the power supply interface by the base device when the hand device is coupled to the base device.

2. A programming devices as set forth in claim 1, wherein the hand device has a data memory which is connected on the one hand to the transmitting and receiving unit and is adapted for autonomous storage of data transmitted from the implant or to the implant and which on the other hand is connected to the data interface of the hand device in such a way that data are to be at least unidirectionally transmitted from the data memory by way of the data interfaces, to the base device when the hand device is coupled to the base device.

3. A programming device as set forth in claim 2, wherein the base device has a printer interfaced or a printers for printing out representations corresponding to the data in the data memory of the hand device.

4. A programming device as set forth in claim 3, wherein the hand device includes a control unity which is connected and adapted to detect a coupled condition of the hand device and in response to detection of the coupled condition to produce a communication between the transmitting and receiving unit of the hand device and the printer interfaces of the base device in such a way that data received on the part of the implant from the transmitting and receiving unit can be represented in real time by way of the printer or the printer interface.

5. A programming devices as set forth in claim 1, wherein the hand device and the base devices each have a respective data transmitting and receiving unit for wireless data exchange between the hand device and the base device.

6. A hand device for a programming devices as set forth in claim 1, wherein the display is formed by a touch-sensitive display screen (touch screen).

7. A hand device or a programming device as set forth in claim 6 wherein the hand device is adapted to make a latching mechanical connection to the base device and includes a release button for releasing the latching mechanical connection.

8. A hand device as set forth in claim 6 wherein the hand device has a shock triggering button which is connected to and co-operates with a control unit of the hand device and the transmitting and receiving unit in such a way that the discharge of a shock by the implant is to be triggered by way of the triggering button.

9. A hand device as set forth in claim 6 further comprising a programming head which is releasably connected to the hand device by way of a flexible electrical feed line and as part of the transmitting and receiving unit of the hand device permits a telemetric communication between the implant and the hand device by applying the programming head to the body of the patient.

10. A base device for a programming device as set forth in claim 1 comprising a main body and a mounting tilting member pivotably connected to the main body for mounting the hand device and adjustment of the angle of inclination of the display of the hand device when the hand device is in a coupled condition.

11. A base device for a programming device as set forth in claim 10 characterized in that the wherein the mounting tilting member has plug connections for a data interface and a power supply interface.

12. A base device for a programming device as set forth in claim 10 characterized in that the main body of the base device includes a printer.

13. A base device for a programming device as set forth in claim 5 comprising a main body and a mounting tilting member pivotably connected to the main body for mounting the hand device and adjustment of the angle of inclination of the display of the hand device when the hand device is in a coupled condition.

14. A base device for a programming device as set forth in claim 13 wherein the mounting tilting member has plug connections for a data interface and a power supply interface.

15. A base device for a programming device as set forth in claim 13 wherein the main body of the base device includes a printer.
